(19) United States
(12) Patent Application Publication
(10) Pub. No.: US 2003/0120125 A1
(43) Pub. Date: Jun. 26, 2003

(54) Ligated platinum group metal catalyst complex and improved process for catalytically converting alkanes to esters and derivatives thereof

(76) Inventors: Roy A. Periana, Los Angeles, CA (US); Douglas J. Taube, Hayward, CA (US); Scott Gamble, San Leandro, CA (US); Henry Taube, Stanford, CA (US)

Correspondence Address: Jill A. Jacobson, Morrison & Foerster LLP, 755 Page Mill Road, Palo Alto, CA, 94304-1018, US

(21) Appl. No.: 10/269,453

(22) Filed: Oct. 11, 2002

Related U.S. Application Data
(63) Continuation of application No. 08/637,067, filed on Apr. 24, 1996.

(51) Int. Cl. C07C 5/00; C07C 331/00

(52) U.S. Cl. 585/500; 585/733; 560/302; 560/304

(57) ABSTRACT

This invention is an improved process for the selective oxidation of lower alkane starting materials (such as methane) into esters and, optionally, into various derivatives (such as methanol) in oxidizing acidic media using a stable platinum group metal ligand catalyst complex at elevated temperatures and to a class of novel platinum group metal ligand complexes employed bidiazine ligands, which are sufficiently stable in the oxidizing acidic media at elevated temperatures to be effective catalysts in the alkane conversion reaction.

### FIELD OF THE INVENTION

This invention relates to an improved process for converting lower alkanes into their corresponding esters using ligand-assisted noble or platinum group metal catalysts and to novel ligated platinum group metal catalysts which are useful in catalyzing the alkane conversion reaction. The process of the invention also includes additional and optional conversion steps whereby the ester product may be converted to other intermediates or derivatives, such as an alcohol or alkyl halide, which, in turn, can be converted to liquid hydrocarbons such as gasoline. More particularly, the invention is directed to an improved process for the selective oxidation of lower alkane starting materials into their corresponding esters and, optionally, into various derivatives (such as methanol) in oxidizing acidic media using a stable platinum group metal ligand catalyst complex at elevated temperatures and to a class of novel platinum group metal ligand complexes which are sufficiently stable in the oxidizing acidic media at elevated temperatures to be effective catalysts in the alkane conversion reaction.

### BACKGROUND OF THE INVENTION

Viable catalytic processes for the oxidative conversion of lower alkanes to useful, more reactive products, such as mono- or poly-hydric alcohols or alkyl halides, which, optionally, may be subsequently converted to higher molecular weight, normally liquid hydrocarbons, such as gasoline, have long been a desired objective in the chemical and petroleum processing industries. In the case of the natural gas industry sector such a catalytic process could enable natural gas or methane produced at remote locations to be converted into a more readily transportable liquid such as methanol, which, in turn, could be used directly as a chemical feedstock or converted to a liquid hydrocarbon such as gasoline by known processing techniques. For other lower alkanes such as ethane, a direct catalytic oxidation which affords a poly-hydric alcohol—e.g., ethylene glycol-could be an attractive alternative to conventional processes which employ olefinic starting materials.

A threshold problem in devising a catalytic process for the partial oxidation of alkanes is the non-reactive nature of the alkane C-H bond and the difficulty in finding a catalytic substance which will promote activation of, and subsequent reaction at, one or more of the C-H bonds of the alkane reactant without also catalyzing complete oxidation of the alkane in question—e.g., methane to CO₂. This threshold problem has been solved, to at least some degree, by the catalytic process described in U.S. Pat. Nos. 5,233,113 and 5,306,855 granted in the name of some of the present inventors, wherein it is taught that high yield, selective oxidation of methane to methyl esters (as well as other hydrocarbons containing C—H bonds) can be obtained with certain classes of metal catalysts in the presence of strongly acidic, oxidizing media. In particular, the aforementioned U.S. patents teach that a class “B” metal from the Mendeleev table and/or Pearson “soft” or borderline metal cations can be employed in catalytic amounts in strong, oxidation resistant-acid media together with an oxidizing agent to convert alkanes, such as methane, to alkyl esters or partially oxidized derivatives thereof. Among the soft, group B metals and metal ions disclosed in the U.S. Pat. Nos. 5,233,113 and 5,306,855 as suitable catalysts are cations of the group VIII noble metals or platinum-group metals, i.e., Pd, Pt, Rh, Ir, Ru and Os, albeit best catalytic activity is ascribed to mercury (Hg).

Cations of platinum group metals, as described in the aforementioned U.S. Pat. Nos. 5,233,113 and 5, 306,855, are good oxidants and can be quite efficient in oxidation reactions of alkanes and other hydrocarbons. However, a notorious problem with these metal ions is their tendency toward catalyst deactivation via irreversible reduction, followed by precipitation of the metallic form of the noble metal. This is because the bulk metal form is the thermodynamically preferred form for the platinum group or noble metals at usual reaction temperatures. This characteristic of the noble metals is the underlying reason for their “noble” character and well-known resistance to corrosion.

In addition to this issue of catalyst loss, a further complication is that the dispersed, metallic forms of these noble metals are well known to be good catalysts for combustion of hydrocarbons. Consequently, the formation of the bulk metal can catalyze unselective oxidation reactions in cases where the intermediate oxidation products are desired. As a result, reaction selectivity tends to be inversely proportional to turnover number. Thus, under stoichiometric reaction conditions where the Pt cation is used as a stoichiometric oxidant, selectivities to methyl esters above 75% can be observed in a typical reaction between methane and H₂Pt(OH)₆ in hot concentrated sulfuric acid so long as all the Pt cations are not consumed. Under these conditions the platinum largely exists as soluble cations which are active for selective oxidation to methyl bisulfate. However, under the conditions where Pt cations are utilized catalytically with increased turnover number, precipitation of bulk metal becomes prevalent and reaction selectivity to methyl bisulfate rapidly drops to the point where CO₂ is the primary carbonaceous product. These issues are the primary basis for the lack of more extensive use of the noble metal cations as catalysts in selective oxidation reactions. Such a deactivation pattern is well documented in the oxidation of ethylene to acetaldehyde catalyzed by Pd(II).

As pointed out above, the second of the cited patents, U.S. Pat. No. 5,306,855, teaches that Ig(II) is the most effective catalyst for the oxidation of methane to methanol in oxidizing, strongly acidic media. In this case, the issue of loss of metal ion by reduction to bulk metal is mitigated because the bulk metal form of Hg is not noble and the cationic state is thermodynamically favored over the metallic state. However, this metal suffers from disadvantages that in sulfuric acid solvents containing free SO₃, a major side product, methane sulfonic acid is produced. The noble metals do not suffer these disadvantages, but have not been used because of the issues of catalyst deactivation and poor selectivity, as discussed above. Thus, it would be advantageous to address the issue of bulk metal formation in the use of the noble metals.

One possibility for allowing the use of noble metals in the alkane oxidation reaction is to modify the reaction system to permit dissolution and reoxidation of the metallic form of the noble metal, and/or to prevent the formation of the metallic metal. In certain cases, this can be accomplished by the use of ligands that stabilize the ionic forms of the metals. Thus, in the case of Pd cation catalysts for olefin oxidation to ketones (the Wacker process), chloride ions are added to stabilize the Pd catalysts in the active, cationic state. Other ligands have been investigated in that system, but in general chloride has been found to be the most ideal ligand because of the resulting stability and high efficiency of the catalytic system. Organic-type ligands, such as amines, phosphines, thiols, alcohols, bromides, iodides, cyanides, etc., are not used because they are not as efficient as chloride and can be destroyed by the oxidizing or acidic conditions of the reaction.

On the basis of the chloride stabilization of Pd(IT) in Wacker chemistry, the use of chloride for the stabilization of platinum in hot, concentrated sulfuric acid was examined for alkane oxidation. Alkanes are much less reactive than olefins, due in large part to the much poorer ability of alkanes to coordinate to the metal center. Coordination of the alkane is one of the basic requirements for efficient catalysis. Consequently, it is generally found that the addition of most species that coordinate to the active catalyst can inhibit reactivity by competitive binding and prevention of alkane coordination. This was found to be the case when the use of Wacker-type conditions for the oxidation of alkanes in oxidizing, strongly acidic media was examined. Thus, addition of chloride to solutions of palladium or platinum sulfate in sulfuric acid resulted in complete inhibition of reactivity with methane. Consistent with the weak coordinating power of methane compared to that of chloride, the addition of chloride resulted in tight binding to the noble metal cations, precipitation of the cations as the polymeric metal chlorides, and loss of catalytic activity.

The reaction system disclosed in the aforementioned U.S. patents for the oxidation of methane to methyl esters is both oxidizing and strongly acidic. Under these conditions, it is very challenging to find ligands that will form a metal-ligated catalyst with platinum group metals which will be stable for useful periods of time, thereby allowing for reaction with alkanes. Such ligated metal catalysts can be destroyed by rupture of the metal-ligand bond. In oxidizing, acidic media, this can readily occur either through oxidation or protonation of the ligand at the site(s) required for binding. While preventing oxidation of the ligands represents a formidable task, preventing protonation and loss of the ligand can be even more challenging. Protonation reactions are quite facile in acidic media, such as concentrated sulfuric acid, where the availability of protons is very high. In general, ligands coordinate to noble metal cations via donation of electron lone pairs, forming dative bonds with the metal. Thus, protonation of the ligand's lone pair could result in irreversible loss of the ligand and concomitant loss of stabilization of the metal cation. The proton activity of concentrated sulfuric acid is very high, binding to most species that exhibit any degree of basicity. In the case of ligands that bind through basic atoms, such as heteroatoms with electron lone pairs (e.g., O, N, S, and P), protonation is expected to be complete and rapid. The infinitesimally small amounts of the unprotonated form that would exist in strongly acidic media would not effectively stabilize the metal cation. Thus, it would not be obvious to one skilled in the art that the use of ligands could or would provide a solution to the rapid loss of catalytic activity which occurs when platinum group metals are employed as catalysts in the conversion of alkanes to esters or other partially oxidized derivatives in oxidizing acidic media.

### SUMMARY OF THE INVENTION

It has now been found that platinum group metal catalysis of the partial oxidation of a lower alkane reactant to form an ester in oxidizing, strongly acidic media can be substantially enhanced by employing a platinum group metal-ligand complex wherein the ligand employed is a heteroatom-containing ligand which forms a mono-dentate or poly-dentate complex with the platinum group metal and the complex so formed is stable in the strong acid reaction media for at least ten minutes at temperatures of at least about 180° C. Stability in this case refers to kinetic stability in the acidic reaction media in that the platinum group metal catalyst complex continues to exist in its catalytically active form in sufficient amounts to catalyze the partial oxidation reaction at useful reaction rates rather than becoming unavailable to catalyze the reaction through a combination of insolubility in the reaction media or loss of structure through protonation and/or oxidation resulting in decomposition of the catalytically active species. By employing the above-described platinum group metal ligand complexes it has been found, surprisingly, that the resulting catalyst is sufficiently active that practically useful yields of ester reaction product can be obtained with mono-dentate or poly-dentate heteroatom-containing ligands which impart the above-described minimum level of stability on the catalyst complex in the acidic, oxidizing reaction media at reaction temperatures.

Accordingly, in its broadest terms, the invention is an improved process for partial oxidation of a lower alkane to form an ester which comprises contacting the lower alkane, an oxidizing agent, a strong acid and a catalyst comprising a catalytic amount of a platinum group metal stabilized with a heteroatom-containing ligand, which forms a mono-dentate or poly-dentate ligand complex with the platinum group metal, said complex being stable in the strong acid for at least about ten minutes at temperatures of about 180° C. and said contacting occurring at esterification conditions to produce a lower alkyl ester of the acid in a molar amount greater than the molar amount of catalytic metal present. It is important to note that while it is expected that the alkyl esters will be the product of the reaction, that formation of protonated alcohols can also be expected to be products of the reaction of alkanes, strong acids and an oxidizing agents and, therefore, in the process of the invention protonated alcohols are considered to be equivalents of the ester products. In other broad aspects, the process of the invention includes subsequent process steps where the alkyl ester product of the partial oxidation is reacted with a nucleophile, such as H₂O or HCl, to yield a functionalized derivative, e.g., an alcohol or alkyl chloride, of the lower alkane starting material and where, optionally, the functionalized derivative of the lower alkane is catalytically converted into a higher molecular weight hydrocarbon.

An additional aspect of the invention is directed to a novel class of ligated platinum group metal catalyst complexes which exhibit high levels of catalytic activity in the acidic, oxidizing reaction media employed in the process of the invention. These novel catalyst compositions comprise a catalytically-active, platinum group metal/ligand complex of the formula MLₘXₙ wherein M is a platinum group metal, L is a bidiazine ligand, optionally substituted with one or more hydrocarbyl groups or substituted hydrocarbyl groups, or a substituent selected from —SO₃OH and fluoride or any mixture thereof, X is an oxidation resistant anion selected from halide, hydroxide, sulfate, bisulfate, nitrate and phosphate or the conjugate anion base of the strong acid reactant, m is 1 or 2 and n is an integer of 1 to 8 depending on the oxidation state of the platinum group metal.

### DESCRIPTION OF THE INVENTION

The process of the invention essentially parallels the step-wise process disclosed in U.S. Pat. Nos. 5, 233,113 and/or 5,306,855, both of which are herewith incorporated by reference, with the exception of the catalyst employed in the first (or ester-forming) step of the process described therein. As pointed out in the reference patents, the first step of the process involves contacting a lower alkane with an acid and an oxidizing agent in the presence of a catalyst, in this case a ligated platinum group metal catalyst complex, at elevated temperatures to afford the alkyl ester product.

The catalyst employed in the first or ester-forming step of the process of the invention is suitably a platinum group metal stabilized with a heteroatom-containing ligand which forms a mono-dentate or poly-dentate ligand complex with the platinum group metal, said complex being stable in the strong acid employed as the solvent for the ester-forming step for at least about ten minutes at temperatures of about 180° C. In this regard, it has been found that platinum group metal ligand complexes which exhibit substantial instability and loss of catalytic activity in the presence of the strong acid at about 180° C. (which is typically the low end of the temperature range for the ester-forming reaction) in less than about 10 minutes of contact time do not afford a sufficient space-time yield of ester product to be useful in industrial scale processes. Preferably, the catalyst complex is stable in the strong acid for at least about 30 minutes and most preferably for greater than two hours. The mono-dentate or poly-dentate, preferably bi-dentate, ligand employed in the catalyst complex is suitably a heteroatom-containing ligand which binds the platinum group metal through one or more nitrogen, sulfur or phosphorus atoms or mixtures thereof—e.g., phosphines, organo-phosphorus compounds, amines and heterocyclic organic compounds containing ring nitrogens and/or sulfur atoms. Preferably, the stabilizing ligand for the catalyst complex is a heteroatom-containing ligand where the heteroatom is nitrogen which forms a bi-dentate ligand complex with the platinum group metal. While, in principle, any platinum group metal may be employed in the catalyst complex used in the ester-forming reaction-e.g., Pt, Pd, Rh, Ir, Rh and Os or mixtures thereof-it is preferred that the platinum group metal be selected from Pd and Pt or mixtures thereof with Pt being most preferred from a catalytic activity standpoint.

Preferably, the catalyst complex employed in the ester-forming reaction is a platinum group metal ligand complex of the formula MLₘXₙ wherein M is a platinum group metal, L is a bidiazine ligand, optionally substituted with one or more hydrocarbyl groups or substituted bydrocarbyl groups, or a substituent selected from —SO₃H and fluoride or any mixture thereof, X is an oxidation-resistant anion selected from halide, hydroxide, sulfate, bisulfate, nitrate and phosphate or the conjugate anion base of the strong acid reactant, m is 1 or 2 and n is an integer of 1 to 8 depending on the oxidation state of the platinum group metal employed. When M is platinum, X is preferably 1, 2, 3 or 4 and, most preferably, 1 or 2. These preferred platinum group metal ligand complexes are believed to be novel and, therefore, comprise another aspect of this invention. Of the novel catalyst composition described by the above formula, it is particularly preferred that M represent platinum and L represent a bidiazine ligand of the formula:

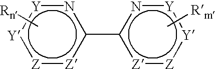

wherein Y, Y′, Z and Z′ are nitrogen or carbon with the proviso that one of Y, Y′, Z and Z′ must be nitrogen and the remainder of Y, Y′, Z and Z′ must be carbon, R and R′ are hydrogen, hydrocarbyl, substituted hydrocarbyl, fluoride or —SO₃H and m′ and n′ are 0, 1, 2 or 3. Platinum catalyst complexes ligated to bidiazine ligands of the above formula possess unique stability in the strong acid media at 180° C. or higher with essentially no loss of catalytic activity being observed in residence times ranging from greater than two hours to several days. Exemplary of suitable bidiazine ligand compounds in this preferred class are the following bidiazine compounds (which may be optionally substituted as set forth above):

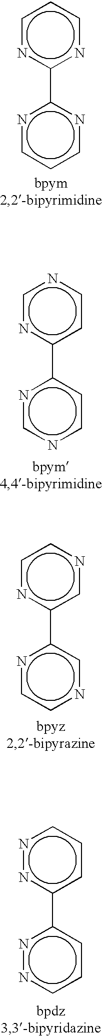

Most preferred are catalysts wherein M represents platinum in the formula given above, L is a 2,2′-bipyrimidine (preferably unsubstituted) and X is a halide selected from chloride, bromide and iodide. In these most preferred catalyst compositions m is 1 and n is 2.

The catalyst complexes of the invention can be prepared by any conventional method for preparing such metal ligand complexes. Suitably, the catalyst complexes are separately prepared by mixing, in appropriate molar ratios, a platinum group metal (in compound or bulk metal form); a ligand compound and an inorganic salt containing the oxidation-resistant anion in an aqueous or weakly acidic media to form the complex which can then be added to the acidic oxidizing reaction media used in the ester-forming reaction. In this regard, in addition to bulk metal form which is suitably a finely divided dispersion of metal, the platinum group metal may be added in the form of a soluble salt compound—e.g., a halide or nitrate, or as an oxide or hydroxide. The inorganic salt employed is suitably an alkali or alkaline earth metal salt or other salt containing a basic cation—e.g., an ammonium salt. It is also convenient to add the platinum group metal in the form of a salt where the anion is one of the oxidation resistant anions set forth for X in the formula given above—e.g, halide, hydroxide, sulfate, bisulfate, nitrate or phosphate and thereby avoid the need for separate addition of an inorganic salt component.

In a preferred aspect of the invention, it has been found that the preferred platinum group metal ligand complex can be most conveniently prepared by adding the catalyst components directly to the strong acid media employed in the ester-forming reaction and allowing the catalyst to form in situ in the reaction zone or vessel used to partially oxidize the lower alkane to the corresponding ester. That is, in this preferred aspect, the active catalyst is prepared in situ in the oxyester-forming reaction zone by mixing the platinum group metal in bulk or compound form, preferably a compound of platinum, a bidiazine compound and an inorganic salt containing the oxidation resistant anion in the strong acid employed as the reaction media for the ester-forming reaction prior to introduction of this lower alkane reactant. In any case whether this catalyst complex is prepared separately or formed in situ, the molar ratios of platinum group metal: bidiazine ligand: inorganic salt components suitably used in the preparation are about 1-2:0.5-1:1-2 dependent on the concentration of strong acid in the reaction zone temperatures and the nature of the ligand employed. For platinum ligand catalyst complexes formed in situ using a 2,2′-bipyrimidine ligand and NaCl as the source of oxidation-resistant anion, the optimum molar ratio of Pt: Ligand: Cl is about 1:0.75:2 for highest catalytic activity in 100% H₂SO₄ at 220° C. in the partial oxidation of methane.

It has also been found that the catalytic activity of the platinum group metal catalyst complex in the ester-forming reaction can be enhanced by the addition of a co-catalyst or oxidation synergist comprising a halide ion or an inorganic salt of tellurium or antimony or mixtures thereof. While not wanting to be bound by any theory, it appears that the platinum group metal catalyst operates in two distinct steps—i.e., a C—H bond activation step which is rapid and an oxidation step which appears to be rate limiting—and the presence of the co-catalyst increases the rate of oxidation of the oxidation step and therefore the rate of the catalytic cycle increases. In cases where a platinum group metal ligand catalyst of the formula MLₘXₙ as given above, is used wherein X is halide, the co-catalyst should be selected from tellurium and antimony salts to maximize the benefit obtained from the co-catalyst. In this regard, preferred co-catalysts include Te(IV) and Te(VI) salts, most preferably, Te halide salts—e.g., TeCl₄, TeCl₅ and TeBr₄. The amount of co-catalyst which is suitably employed relative to the amount of catalyst complex present can vary over wide limits depending on the other reaction conditions used but typically ranges between about 0.5 to 4 moles of co-catalyst per mole of platinum group metal catalyst complex present. Preferably, the co-catalyst is present at from about 1 to about 2 moles per mole of catalyst complex employed in the reaction zone.

Lower alkanes which may be suitably employed as starting materials for the ester-forming reaction include C₁ to C₈ straight or branched-chain alkanes—e.g., methane, ethane, propane, isobutane, hexane and heptane. Preferably, the lower alkane starting material is a straight chained alkane of 1 to 4 carbon atoms, that is, methane, ethane, propane or butane, and, most preferably, the alkane starting material is methane including impure forms of methane such as that found in natural gas reservoirs.

The oxidizing agent employed in the ester-forming reaction may be a strong oxidant such as those disclosed in the referenced U.S. Pat. Nos. 5,233,113 and 5,306,855—e.g., HNO₃, perchloric acid, peroxy compounds (1202, CH₃CO₃H, K₂S₂O₈), hypochlorites (such as NaOCl), O_{2, O}₃, SO₃, NO₂, and cyanogen, as well as a variety of other oxidizing substances having redox potentials greater than 0.3 volts—e.g., quinones, halogens, selenium cations, tellurium cations and the like. Preferred oxidants from a cost of materials, availability and effectiveness standpoints include SO₃, H₂SO₄ and O₂ while oxidants which can be recycled with O₂—e.g., SO₃, H₂O₂, quinone and cations of selenium and/or tellurium—are also advantageous. In carrying out the process of the invention, the oxidant can be added to the ester reaction zone before, or after, or during the addition of the alkane starting material. The amount of oxidizing agent employed is typically at least stoichiometric with the amount of alkane starting material added to the reaction zone.

Similarly, the acid employed as the reaction medium or solvent in the ester-forming reaction may be any of the acids described in the referenced U.S. patents (see above), including organic or inorganic acids such as HNO₃, H₂SO₄, CF₃CO₂H, CF₃SO₃H, H₃PO₄, HCl, HF, HPAs (heteropolyacids), B(OH)₃, (CF₃SO₂)₂HN, (CF₃SO)₃CH or the like, anhydrides of these acids such as H₄P₂O₇, H₂S₂O₇ or the like and mixtures of two or more of these acids and anhydrides and mixtures of acids with Lewis acids such as CH₃CO₂H/BF₃,H₃PO₄/BF₃, H₃PO₄/SbF₅, HF/BF3. The preferred acids are strong acids having pKₐs of less than 2.0 with H₂SO₄ and CF₃SO₃H being particularly preferred. Without being bound to theory, it is believed that the function of the acid is to generate an alkyl compound containing an electron withdrawing group such as —OSO₃H, —OSO₂CF₃ or —OH₂⁺. In general, it is felt that the function of the electron withdrawing group is to “protect” the alkyl group from over-oxidation by electrophilic catalysts. This form of “protection” is similar to that observed when an aromatic ring in nitrated; due to the electron withdrawing characteristics of the nitro group subsequent oxidation by electrophilic species is inhibited and the nitro-arene is “protected.” As noted in the referenced patents (see above), the acid is desirably used in excess since it can act both as the reaction medium and as a reactant in the process, that is, the acid contributes the anion to form the ester on oxidation of the alkane. In this regard, the acid employed is desirably oxidation-resistant in that it is not itself oxidized by the platinum group metal complex in the noted reaction medium. In a most preferred case, H₂SO₄ is employed as the reaction medium together with an oxidizing agent selected from SO₃, O₂ and H₂SO₄. In this latter case, H₂SO₄ functions both as the acid and the oxidant. In this regard, a key advantage of the platinum group metal catalysts, especially platinum, over Hg(II) of that these catalysts do not produce alkane sulfonic acid from alkane in the presence of free SO₃.

The ester-forming reaction can be carried out either batchwise or continuously using processing methods or techniques which are well known in the art. The amount of catalyst complex employed must be at least a catalytic amount with amounts ranging between about 50 ppm and 1.0% by mole of the total liquid present being effective. Further the temperatures of the ester-forming reaction is typically above 50° C. and preferably between 95° C. and 250° C. with temperatures in the range of about 180° to 230° C. being most preferred. When methane is the alkane reactant, it is added at a pressure above about 50 psig, preferably, above about 450 psig. In the ester-forming reactions these conditions result in the production of the alkyl ester of the acid in a molar amount greater than the molar amount of the catalyst complex charged to the reactor. In fact, with the catalyst complexes of the invention, alkane (methane) conversions of greater than 80% at selectivities of 90% and space time yields of 10⁻⁷ mol/cc.sec at catalyst turnovers greater than 300 are achievable.

In the optional steps of the process, as described in the referenced patents (see above), a nucleophile is reacted with the ester to form a functionalized derivative of the lower alkane and the functionalized derivative is then catalytically converted to a comparatively higher molecular weight hydrocarbon. In the first optional step, the ester may be reacted directly with a nucleophilic substance or, optionally, the ester may be recovered from the ester-forming reaction by flashing or distillation and then reacted with a nucleophilic substance such as water or a hydrogen halide to produce the functionalized derivative of the alkane starting material. For example, in the case of a methyl ester prepared using methane as the starting alkane, the functionalized derivative is methanol if this nucleophile is H₂O; methyl halide, if the nucleophile is a hydrogen halide such as HCl, HBr, or Hl; methyl amine, if the nucleophile is NH₃; or a methyl thiol, if the nucleophile is H₂S or acetonitrile if the nucleophile is HCN. In general a variety of the other functionalized derivatives can be generated from the original methyl ester by reaction with other nucleophiles, e.g., other esters such as methyl triflouroacetate if the nucleophile is triflouroacetic acid. It should be understood that the word “nucleophile” is used generally in this context and to one skilled in the art many such exchange reactions can be considered. These reactions proceed readily to completion. An excess of the nucleophile is desirable. The preferred nucleophile is H₂O since it may also be produced in the ester-forming reaction. The product methanol may be used directly, or may be converted to a variety of hydrocarbons in the subsequent optional step.

The subsequent optional process step includes conversion of the functionalized alkane derivative—e.g., methanol to a longer chain or higher molecular weight hydrocarbon.

Suitable processes for converting methanol and other methyl intermediates to higher molecular weight hydrocarbons are found in U.S. Pat. Nos. 3,894,107 and 3,979,472 to Butter et al. Butter shows the production of olefinic and aromatic compounds by contacting the methyl intermediate with an aluminosihcate catalyst, preferably HZSM-5, at a temperature between 650°-1000° F.

Similarly, Butter suggests a process using a preferable catalyst of antimony oxide and HZSM-5 at a temperature between 250°-700° C.

The ZSM-5 zeolite has been disclosed as a suitable molecular sieve catalyst for converting methyl alcohol into gasoline range hydrocarbons. See, for instance, U.S. Pat. Nos. 3,702,886 to Argauer et al. and 3,928,483 to Chang et al.

Other processes include those described in U.S. Pat. No. 4,373,109 to Olah (bifunctional acid-base catalyzed conversion of methanol and other methyl intermediates into lower olefins); U.S. Pat. No. 4,687,875 to Currie et al. (metal coordination complexes of heteropolyacids as catalyst for converting short chain aliphatic alcohols to short change hydrocarbons); U.S. Pat. No. 4,524,234 to Kaiser (production of hydrocarbons, preferably from methanol using aluminophosphate molecular sieves); and U.S. Pat. No. 4,579,996 to Font Freide et al. (production of hydrocarbons from C₁ to C₄ monohaloalkanes using layered clays); etc. Each of the above is potentially suitable for the second optional step of this process and their contents are incorporated herein by reference.

### EXAMPLES

The following examples demonstrate some of the advantages achieved with the ligated platinum group metal catalyst complexes in the process of the invention.

### General Procedures

A. Analysis of Reaction Products

The reaction products were analyzed by gas chromatography (GC), high-pressure liquid chromatography (HPLC), and nuclear magnetic resonance spectroscopy (NMR). The gas phase of the reactions of methane with platinum compounds in sulfuric acid were analyzed by gas chromatography on a Hewlett-Packard 5880 GC fitted with a HayeSep© D packed column and a thermal conductivity detector. The response factors for the gases, Ne, CH₄, CO₂, CO, SO₂, and CH₃Cl, were obtained by the injection of a calibration gas mixture (Alphagaz). Neon was added to the feed methane (3 mole %) as an internal standard.

The liquid phase of the reaction was analyzed by both HPLC and NMR. For HPLC analysis, the reaction solution was hydrolyzed by the addition of 1 mL reaction solution to 3 mL distilled water and heated to 95° C. for 2 hours. The hydrolyzed solution was injected onto a Hewlett-Packard 1050 HPLC equipped with a Aminex© HPX87H ion exclusion column and a refractive index detector. The eluant was 0.01% H₂SO₄ in water. The response factors for the soluble organic products, methanol, acetic acid, formic acid, and formaldehyde, were measured from standard solutions.

The reaction solutions were also analyzed by multinuclear NMR (¹H and ¹³C). The concentration of the products in the neat reaction solutions were measured by NMR using acetic acid as an internal standard.

B. Synthesis of Catalysts

The catalysts, including Pt(bpym)Cl₂ were synthesized according to general literature procedures (Kiernan, P. M., Ludi, A., J. C. S. Dalton, 1978, 1127). In short, K₂PtCl₄ and the appropriate ligand added in a stoichiometric ratio, were added to distilled water and allowed to stir for several hours. During this time, the initially orange solution became cloudy and a precipitate formed. When the solution had become void of color, the reaction was filtered giving a powder. In most cases the solid was air dried and used. In the case of Pt(bpym)Cl₂, the solid formed a hydrate, Pt(bpym)Cl₂ 0.5 H₂O. The solid was dehydrated by adding the dark green solid to acetone resulting in an orange solid.

C. Reaction Procedures

The reactions of the platinum catalyst complexes with the alkane reactant in sulfuric acid were conducted in either a 300 cc autoclave or a 100 cc Parr bomb. Mass balance and kinetic studies with in situ sampling were run in the 300 cc autoclave while batch reactions were performed in the Parr bomb.

The 300 cc autoclave (Autoclave Engineers) was constructed of Hasteloy C. The internal parts were tantalum (stir shaft, impeller and baffle) or covered with glass. The reaction was stirred by an external Magna drive stirrer connected to an impeller. The reaction solution was loaded into a glass liner which fit snugly into the reactor body. Methane was fed into the reactor using a high-pressure feed cylinder. The amount of methane fed into the reactor was measured by the pressure drop in the feed cylinder.

The ester-forming reactions in sulfuric acid were run at reaction temperatures between 180°-220° C. for 1 to 6 hours. Reactions conducted in the 300 cc autoclave were typically run in the batch mode. At the end of the reaction, the reactor was cooled to room temperature by the use of a water jacket, and the gas phase bled to an evacuated cylinder. The gas was analyzed by GC. A second venting of the reactor head space into an evacuated cylinder was conducted so that the final reactor pressure was less than 500 torr. The second venting was performed to remove most of the soluble gases from the reaction solution. The gases from the second venting were also analyzed by GC. The reaction solution was analyzed by HPLC and NMR.

The carbon mass balance of the reaction was measured in two separate ways, by the use of neon as an internal standard, and by measuring the amount of the exit gases using the ideal gas law. Typically, both methods gave carbon mass balance values of greater than 95%.

Reactions were also run on a smaller scale in 100 cc Parr bombs. These reactions were heated by an external oil bath and stirred by using a Teflon® stir-bar driven by an external magnetic stirrer. The reaction solution volumes, typically 5 ml, were added to a glass vial equipped with a weep hole. Analysis of the gas phase was by GC, and the solution phase by HPLC and NMR. Carbon mass balance values were not obtained in the Parr bomb. Selectivities were determined from the observable products, primarily methanol and CO₂.

Several specific examples of experimental methods for oxidation of methane and ethane in sulfuric acid are described below.

### Example 1

This example describes the oxidation of methane at high pressure using a platinum 2,2′-bipyrimidine iodide catalyst complex (Pt(bpym)I₂) in 100.5% H₂SO₄. The experiment was conducted in a 300 cc autoclave using the procedure described above.

A mixture of Pt(bpym)I₂ (3.64 g, 6.0 mmol) and H₂SO₄ (100.5%; 120 mL) was placed in a glass lined autoclave. The reactor was flushed with nitrogen, warmed to 200° C., then pressurized to 500 psi with methane. After 240 min at 200° C., the reaction was halted, the reactor was cooled and vented, and the off-gases were collected. Analysis of the gas phase by GC indicated 14.314 mmol CO₂ and 163.848 mmol SO₂.

The reaction solution and reactor wash solutions were analyzed by removing 1 mL aliquots of each, diluting in 3 mL H₂O, sealing in sample vials which were placed in a heater block at 95° C. for 120 mins. After hydrolysis, the solutions were cooled, centrifuged, and analyzed by HPLC. The HPLC traces indicated a methanol concentration of 860.4 mM in the original reaction solution, and a total of 103.252 mmol methanol. The selectivity to methanol was 80.04%, with a methanol yield of 71.17% based on a methane conversion of 88.92%. The carbon mass balance was 92.53%. Selectivity is defined as percent selectivity to methanol product determined by dividing the moles of methanol found in the final reaction product by the moles of methane consumed in the reaction times 100. Percent conversion is calculated as moles of methane consumed divided by moles of methane charged times 100 and percent yield is determined by multiplying selectivity times conversion.

### Example 2

Using the procedure described in Example 1, a series of experiments were conducted comparing the mercury catalyst of the prior art with the ligated catalyst of the invention in the oxidation of methane to methanol. These experiments were conducted in a 300 cc autoclave. The concentration of the catalysts were 50 mM for the platinum catalysts and 100 mM for HgSO₄. The concentration of methanol produced in the experiment in which the catalyst was generated in situ (H₂Pt(OH)₆+bpym+TeCl₄) was 1.05 M. The results are given in Table 1 below where percent selectivity (to methanol) and percent methane conversion is as defined in Example 1.

TABLE 1

| | | Temp | CH₄ | CH₃OSO₃H | |
|---|---|---|---|---|---|
| Catalyst | H₂SO₄ | (° C.) | Conversion | Selectivity | Yield |
| HgSO₄ | 100% | 180 | 50% | 86% | 43% |
| Pt(bpym)Cl₂ | 100.5% | 200 | 78% | 72% | 56% |
| Pt(bpym)I₂ | 100.5% | 200 | 93% | 76% | 70% |
| H₂Pt(OH)₆ + | 102.3% | 200 | 90% | 79% | 71% |
| bpym + TeCl₄ | | | | | |

### Example 3

This example describes the oxidation of methane at high pressure using a platinum 2,2′-bipyrimidine bromide catalyst complex (Pt(bpym)Br₂) in 96% H₂SO₄. The reaction was conducted in a 100 mL Parr reactor.

A mixture of Pt(bpym)Br₂ (0.128 g, 0.25 mmol) and H₂SO₄ (96%; 5 mL) was placed in a glass vessel with a stir bar, which was then placed in a Parr bomb reactor. The reactor was flushed with methane, then pressurized to 400 psi with methane and placed in an oil bath. The bath was warmed to 215° C. with stirring. After 120 min at 215° C., the pressure had risen to 610 psi. At this point the oil bath was removed and the reactor was cooled in a water bath. After cooling, the reactor was vented and the off-gases were collected and analyzed by GC. The GC trace indicated 0.152 mmol CO₂ and 1.787 mmol SO₂.

A 1 mL aliquot of the reaction solution was diluted in 3 mL H₂O, and sealed in a sample vial which was placed in a heater block at 95° C. for 120 mins. After hydrolysis, the solution was cooled, centrifuged, and analyzed by HPLC. The HPLC trace indicated methanol at a concentration of 487.0 mM (2.435 mmole) in the original reaction solution.

### Example 4

This example describes the oxidation of methane at high pressure using a platinum ammine chloride catalyst complex (c-Pt(NH₃)₂Cl₂) in 96% H₂SO₄.

A mixture of t-Pt(NH₃)₂Cl₂ (0.175 g, 0.585 mmol) and H₂SO₄ (96%; 5.85 mL) was placed in a glass vessel with a stir-bar, which was then placed in a Parr bomb reactor. The reactor was flushed with methane, then pressurized to 500 psi with methane and placed in an oil bath. The bath was warmed to 180° C. with stirring. After 25 mins. at 180° C., the pressure had risen to 550 psi. At this point the oil bath was removed and the reactor was cooled in a water bath. After cooling, the reactor was vented and the off-gases were collected and analyzed by GC. The GC trace indicated CO₂ (0.052 mmole), SO₂ (4.04 mmole), and CH₃Cl (0.402 mmole).

A 1 mL aliquot of the reaction solution was diluted in 3 mL H₂O, and sealed in a sample vial which was placed in a heater block at 95° C. for 120 min. After hydrolysis, the solution was cooled, centrifuged, and analyzed by HPLC. The HPLC trace indicated methanol at a concentration of 493.5 mM in the original solution (2.887 mmole).

### Example 5

Using the procedure described in Example 1, a series of experiments were conducted comparing the mercury catalyst of the prior art with the ligated catalyst of the invention, PtCl₂, and H₂Pt(OH)₆ in the oxidation of methane to methanol. These experiments were conducted in a Parr bomb using the procedure described in Example 3. The concentration of the catalysts were 25 mM except for PtCl₂ which was 100 mM. The concentration of methanol produced in the experiment in which the catalyst was generated in situ (H₂Pt(OH)₆+bpym+TeCl₄) was 1.05 M. The results are given in Table 2 below where percent selectivity (to methanol) is determined by dividing the moles of methanol found in the final reaction product by the moles of methane consumed in the reaction times 100.

TABLE 2

| Catalyst | Temp (° C.) | Time (min) | [MeOH] | Selectivity |
|---|---|---|---|---|
| HgSO₄ | 180 | 180 | 311 mM | 96% |
| | 220 | 180 | 232 mM | 14% |
| Pt(bpym)Cl₂ | 180 | 180 | 82 mM | 80% |
| | 220 | 180 | 406 mM | 84% |
| Pt(NH₃)₂Cl₂ | 180 | 180 | 304 mM | 85% |
| | 220 | 180 | 179 mM | 62% |
| PtCl₂ | 180 | 30 | 0 mM | 0% |
| | 220 | 150 | 29 mM | 72% |
| H₂Pt(OH)₆ | 180 | 180 | 51 mM | 46% |
| | 220 | 180 | 18 mM | 11% |

### Example 6

This example describes the oxidation of methane at high pressure using a platinum triethyl-phosphine hydrochloride catalyst complex (Pt(PEt₃)₂HCl) in 96% H₂SO₄.

A mixture of Pt(PEt)₂HCl (0.117 g, 0.25 mmol), TeCl₄ co-oxidant (0.107 g, 0.397 mmol) and H₂SO₄ (96%; 5 mL) was placed in a glass vessel with a stir-bar, which was then placed in a Parr bomb reactor. The reactor was flushed with methane, then pressurized to 400 psi with methane and placed in an oil bath. The bath was warmed to 190° C. with stirring. After 120 min. at 190 C, the pressure had risen to 570 psi. At this point the oil bath was removed and the reactor was cooled in a water bath. After cooling, the reactor was vented and the off-gases were collected and analyzed by GC. The GC trace indicated 0.028 mmol CO₂, 0.099 mmol CH₃Cl, and 0.514 mmol SO₂.

A 1 mL aliquot of the reaction solution was diluted in 3 mL H₂O, and sealed in a sample vial which was placed in a heater block at 95° C. for 120 min. After hydrolysis, the solution was cooled, centrifuged, and analyzed by HPLC. The HPLC trace indicated methanol at a concentration of 51.4 mM (0.257 mmole).

### Example 7

This example describes the oxidation of ethane at high pressure using a platinum 2,2′-bipyrimidine chloride catalyst complex (Pt(bpym)Cl) in 102% H₂SO₄.

A mixture of Pt(bpym)Cl₂ (0.212 g, 0.50 mmol) and H₂SO₄ (102%; 5 mL) was placed in a glass vessel with a stir-bar, which was then placed in a Parr bomb reactor. The reactor was flushed with ethane, then pressurized to 250 psi with ethane and placed in an oil bath. The bath was warmed to 150° C. with stirring. After 60 min at 150° C., the pressure had risen to 360 psi. At this point the oil bath was removed and the reactor was cooled in a water bath. After cooling, the reactor was vented and the off-gases were collected and analyzed by GC. The GC trace indicated 0.015 mmol C02 and 0.760 mmol SO₂.

A 1 mL aliquot of the reaction solution was diluted in 3 mL H₂O, and sealed in a sample vial which was placed in a heater block at 95° C. for 120 min. After hydrolysis, the solution was cooled, centrifuged, and analyzed by HPLC. The HPLC trace indicated 1,2-ethane diol at a concentration of 16.6 mM and 1-chloro-2-ethanol at a concentration of 7.6 mM.

### Example 8

This example describes the oxidation of ethane at high pressure using a platinum 2,2′-bipyrimidine sulfate catalyst complex CPt(bpym)SO₄) in 102% H₂SO₄.

A mixture of Pt(bpym)SO₄ (0.112 g, 0.25 mmol) and H₂SO₄ (102%; 5 mL) was placed in a glass vessel with a stir-bar, which was then placed in a Parr bomb reactor. The reactor was flushed with ethane, then pressurized to 250 psi with ethane and placed in an oil bath. The bath was warmed to 150° C. with stirring. After 60 min at 150° C., the pressure had risen to 360 psi. At this point the oil bath was removed and the reactor was cooled in a water bath. After cooling, the reactor was vented and the off-gases were collected and analyzed by GC. The GC trace indicated 0.015 mmol CO₂ and 2.352 mmol SO₂.

A 1 mL aliquot of the reaction solution was diluted in 3 mL H₂O, and sealed in a sample vial which was placed in a heater block at 95° C. for 120 min. After hydrolysis, the solution was cooled, centrifuged, and analyzed by HPLC. The HPLC trace indicated ethanol at a concentration of 32.6 mM and acetic acid at a concentration of 1.8 mM. Subsequent ion chromatography indicated isethionic acid, HOCH₂CH₂SO₃H, in a concentration of 644 mM.

### Example 9

Using the procedure described in Example 3, a series of mono-dentate ligand platinum catalyst complexes were tested for activity for the oxidation of methane to methanol in 96% H₂SO₄. The results are reported in Table 3 below where “selectivity” refers to percent selectivity to methanol product determined by dividing the moles of methanol found in the final reaction product by the moles of methane consumed in the reaction times 100.

TABLE 3

| | [Catalyst] | | | Selec- |
|---|---|---|---|---|
| Catalyst | mM | Time (min) | [MeOH] | tivity |
| Pt(NH₃)₂Cl₂ | 104 | 25 | 493 mM | 99 |
| Pt(NH₂CH₃)₂Cl₂ | 101 | 45 | 75 mM | 62 |
| Pt(NH₂Et)₂Cl₂ | 105 | 32 | 11 mM | 40 |
| Pt(1-Me imidazole)₂Cl₂ | 100 | 45 | 25 mM | 76 |
| Pt(pyridine)₂Cl₂ | 98 | 40 | 16 mM | 24 |

### Example 10

Using the procedure of Example 3, an additional series of platinum catalysts (complexed and uncomplexed) were tested in the oxidation of methane to methanol. The results are given in Table 4 below where the ligands used include en or ethylene diamine, bpy or 2,2′-bipyridine, bpym or 2,2′-bipyrimidine, bpym′ or 4,4′-bipyrimidine, bpyz or 2,2′-bypyrazine, bpdz or 3,3′-bipyridazine. The selectivities were determined as described above in Example 9.

TABLE 4

| | | | | Selec- |
|---|---|---|---|---|
| Catalyst | Temp (° C.) | Time (min) | [MeOH] | tivity |
| Pt(OH)₄ | 180 | 60 | 77 mM | 35% |
| Pt(NH₃)₂Cl₂ | 180 | 25 | 493 mM | 99% |
| Pt(en)Cl₂ | 180 | 60 | 43 mM | 98% |
| Pt(bpy)Cl₂ | 180 | 90 | 0 mM | — |
| *H₂Pt(OH)₆ + bpym | 190 | 120 | 698 mM | 86% |
| *H₂Pt(OH)₆ + bpym′ | 190 | 120 | 174 mM | 70% |
| *H₂Pt(OH)₆ + bpyz | 190 | 120 | 113 mM | 67% |
| *H₂Pt(OH)₆ + bpdz | 190 | 120 | 21 mM | 72% |

*TeCl₄ added to the solvent to aid oxidation

### Example 11

Using the reaction procedure described in Example 1, a comparison was made of a preformed catalyst complex and a catalyst complex formed in situ in the catalytic oxidation of methane to methanol. In this case, the catalyst complex used was Pt(bpym)Cl₂ preformed as described above or formed in situ from the catalyst components bypyrimidine, chloride and platinum or bypyrimidine, sulfate and platinum. The results are shown in Table 5. These reactions were conducted in the 300 cc autoclave. The platinum concentration for these experiments was 50 mM. The reactions were run for 90 minutes at 215° C. under 500 psig CH₄/Ne. The “Pt(bpym)(Cl)(OSO₃H)” catalyst stoichiometry was prepared by adding 25 mM each of Pt(bpym)Cl₂ and Pt(bpym)SO₄.

TABLE 5

| Catalyst | [MeOH] mM Formed |
|---|---|
| Pt(bpym)Cl₂ | 211 |
| H₂Pt(OH)₆ + bpym + 2 NaCl (in situ) | 232 |
| Pt(bpym)(Cl)(OSO₃H) | 203 |
| H₂Pt(OH)₆ + bpym + NaCl (in situ) | 213 |

### Example 12

In a manner similar to that described in Example 11, the catalytic activity of a platinum bipyrimidine chloride catalyst complex in the oxidation of methane to methanol was examined where the catalyst complex was prepared in situ from varying molar ratios of the catalyst components bipyrimidine, chloride and platinum. The effects of these changes on catalytic activity are shown in Table 6 below where the amount of methanol formed (in mM) is given in the tabular columns.

TABLE 6

| | bpym/Pt | | |
|---|---|---|---|
| Cl/Pt | Mole Ratio | | |
| Mole Ratio | 0.5 | 0.75 | 1.0 |
| 0 | 88 mM | 84 mM | 71 mM |
| 1 | 234 mM | 207 mM | 213 mM |
| 2 | 216 mM | 271 mM | 232 mM |

### Example 13

The effects of various co-catalysts on the activity of the catalyst complexes of the invention was evaluated using the general procedure disclosed in Example 3. The experimental results which are given in Table 7 below show the effects of additives such as halides and tellurium salts on methanol productivity. The experiments were run for 120 mins. in 96% H₂SO₄ under 400 psig CH₄/Ne in a Parr bomb. The platinum concentration was 50 mM in each experiment.

TABLE 7

| | | | | Selec- |
|---|---|---|---|---|
| Catalyst | Additive | Temp (° C.) | [MeOH] | tivity |
| H₂Pt(OH)₆/bpym | — | 190 | 66 mM | 89% |
| Pt(bpym)Cl₂ | — | 190 | 106 mM | 82% |
| Pt(bpym)Br₂ | — | 190 | 100 mM | 90% |
| Pt(bpym)I₂ | — | 190 | 239 mM | 96% |
| Pt(bpym)Cl₂ | — | 215 | 371 mM | 84% |
| Pt(bpym)Br₂ | — | 215 | 487 mM | 94% |
| Pt(bpym)I₂ | — | 215 | 636 mM | 90% |
| Pt(bpym)Cl₂ | 200 mM H₆TeO₆ | 190 | 441 mM | 89% |
| Pt(bpym)Cl₂ | 200 mM TeO₂ | 190 | 542 mM | 91% |
| H₂Pt(OH)₆/bpym | 70 mM TeCl₄ | 190 | 698 mM | 86% |

### Example 14

Additional ligated platinum catalyst complexes were tested in the conversion of methane to methanol using the general procedure of Example 3. The catalysts tested and the relevant test conditions and results are listed in Table 8 below.

TABLE 8

| | [Cat.] | % | | [MeOH] | % Sel. to |
|---|---|---|---|---|---|
| Catalyst | mM | H₂SO₄ | Temp. (° C.) | (mM) | MeOH |
| Pt(NH₂CH₃)₂Cl₂ | 101 | 96 | 180 | 75 | 62 |
| Pt(py)₂Cl₂ | 98 | 96 | 180 | 16 | 24 |
| Pt(Melm)₂Cl₂ | 100 | 96 | 180 | 25 | 76 |
| Pt(DDP)Cl₂ | 51 | 101 | 180 | 173 | 84 |
| Pt(pypym-py)Cl₂ | 47 | 96 | 220 | 34 | 70 |
| Pt(Tp)Cl₂ | 52 | 96 | 220 | 98 | 32 |
| Pt(pdtri)Cl₂ | 60 | 98 | 220 | 8 | 47 |
| Pt(tacn)Cl₂ | 56 | 96 | 220 | 26 | — |
| Pt(aquin)Cl₂ | 49 | 96 | 220 | 35 | 39 |
| Pt(biim)Cl₂ | 68 | 96 | 190 | 48 | 29 |
| Pt(pybpym)Br₂ | 50 | 96 | 215 | 391 | 86 |
| Pt(dpbpym)Cl₂ | 50 | 96 | 215 | 10 | 57 |
| Pt(phbpym)Cl₂ | 40 | 96 | 190 | 8 | 35 |
| Pt(TAP)Cl₂ | 25 | 96 | 215 | 22 | 25 |
| Pt(HAT)Cl₂ | 50 | 96 | 190 | 74 | 26 |
| Pt(PEt₃)₂(H)Cl | 50 | 96 | 190 | 51 | 67 |

| | |
|---|---|
| py | pyridine |
| Melm | 1-methylimidazole |
| DPP | 2,3-bis(2-pyridyl)pyrazine |
| Tp | hydrido-tris(1-pyrazolyl)borate |
| pdtri | 3-(2-pyridyl)-5,6-diphenyl-1,2,4-triazine-p,p′-disulfonic acid |
| tacn | 1,4,7-triazacyclononane |
| aquin | 5-aminoquinoxaline |
| biim | 2,2′-biimidazole |
| pybpym | 4-(2-pyridyl)-2,2′-bipyrimidine |
| dpbpym | 4,6-diphenyl-2,2′-bipyrimidine |
| phbpym | 6-phenyl-4-hydxoxy-2,2′-bipyrimidine |
| TAP | 1,4,5,8-Tetraazaphenanthrene |
| HAT | 1,4,5,8,9,12-Hexaazatriphenylene |
| pypym | 2-(2-pyridyl)pyrimidine |

### Example 15

Table 9 lists several experiments investigating the selective oxidation of ethane to ethanol, 1,2-ethane diol, and halide-substituted analogs using the general procedure of Example 7. These experiments were conducted in a Parr bomb using 300 psig CH₃CH₃/Ne (2.99 mol % Ne). The sulfuric acid concentrations, reaction temperatures, and times are listed in the table. The gases, including Ne, O₂, N₂, CH₃CH₃, CO₂, and CH₃CH₂Cl, were collected and analyzed by GC as in the methane experiments. The liquid phase was diluted 1:3 with distilled water, heated to 95° C. for 2 hours to hydrolyze bisulfate esters to alcohols, and analyzed by HPLC. The HPLC was calibrated for ethanol, 1,2-ethane diol, acetic acid, 1-chloro-2-ethanol, and acetaldehyde.

TABLE 8

| | | Temp | Time | | |
|---|---|---|---|---|---|
| Catalyst | Acid | (° C.) | (min) | XCH₂CH₂X | CH₃CH₂X |
| Pt(NH₃)₂Cl₂ | H₂SO₄ (96%) | 150 | 60 | 13 mM | 16 mM |
| Pt(NH₃)₂Br₂ | H₂SO₄ (96%) | 150 | 15 | 40 mM | — |
| Pt(NH₃)₂Br₂ | H₂SO₄ | 150 | 30 | 65 mM | — |
| | (102%) | | | | |
| Pt(NH₃)₂I₂ | H₂SO₄ | 150 | 30 | 140 mM | — |
| | (102%) | | | | |
| Pt(bpym)Cl₂ | H₂SO₄ | 150 | 60 | 25 mM | — |
| | (102%) | | | | |
| Pt(bpym)SO₄ | H₂SO₄ | 150 | 60 | — | 33 mM |
| | (102%) | | | | |

X = Cl, Br, OSO₃H

### Example 16

This example describes the oxidation of methane at high pressure using Pt(NH₂CSCSNH₂)Cl, in 96% H₂SO₄.

A mixture of Pt(NH₂CSCSNH₂)Cl₂ (0.098 g, 0.25 mmol) and H₂SO₄ (96%; 5 mL) was placed in a glass vessel with a stirbar, which was then placed in a Parr reactor. The reactor was flushed with methane, then pressurized to 400 psi with methane and placed in an oil bath. The bath was warmed to 190° C. with stirring. After 120 min at 190° C., the pressure had risen to 620 psi. At this point the oil bath was removed and the reactor was cooled in a water bath. After cooling, the reactor was vented and the off-gases were collected and analyzed by GC. The GC trace indicated 1.545 mmol CO, and 3.369 mmol SO₂.

A 1 mL aliquot of the reaction solution was diluted in 3 mL H₂O, and sealed in a sample vial which was placed in a heater block at 95° C. for 120 min. After hydrolysis, the solution was cooled, centrifuged, and analyzed by HPLC. The HPLC trace indicated methanol at a concentration of 98.4 mM (0.492 mmole) in the original reaction solution.

### Example 17

This example describes the oxidation of methane at high pressure using PtS₂ in 96% H₂SO₄.

A mixture of PtS₂ (0.198 g, 0.76 mmol) and H₂SO₄ (96%; 5 mL) was placed in a glass vessel with a stirbar, which was then placed in a Parr reactor. The reactor was flushed with methane, then pressurized to 440 psi with methane and placed in an oil bath. The bath was warmed to 180° C. with stirring. After 85 min at 180° C., the pressure had risen to 630 psi. At this point the oil bath was removed and the reactor was cooled in a water bath. After cooling, the reactor was vented and the off-gases were collected and analyzed by GC. The GC trace indicated 1.274 mmol CO₂ and 5.422 mmol SO₂.

A 1 mL aliquot of the reaction solution was diluted in 3 mL H, O, and sealed in a sample vial which was placed in a heater block at 95° C. for 120 min. After hydrolysis, the solution was cooled, centrifuged, and analyzed by HPLC. The HPLC trace indicated methanol at a concentration of 69 mM (0.345 mmole) in the original reaction solution.

What is claimed is:

1. A process for partial oxidation of a lower alkane to form an ester which comprises contacting the lower alkane, an oxidizing agent, a strong acid and a catalyst comprising a catalytic amount of a platinum group metal stabilized with a heteroatom-containing ligand, which forms a mono-dentate or poly-dentate ligand complex with the platinum group metal, said complex being stable in the strong acid for at least about ten minutes at temperatures of about 180° C. and said contacting occurring at esterification conditions to produce a lower alkyl ester of the acid or protonated alcohol in a molar amount greater than the molar amount of catalytic metal present.

2. The process of claim 1 wherein the catalyst is a platinum group metal stabilized with a heteroatom-containing ligand where the heteroatom is nitrogen which forms a bidentate ligand complex with the platinum group metal.

3. The process of claim 1 or 2 wherein the catalyst incorporates a co-catalyst selected from halide ions and inorganic salts of tellurium or antimony or mixtures thereof.

4. The process of claim 3 wherein the platinum group metal is selected from platinum and palladium.

5. The process of claim 4 wherein the platinum group metal is platinum.

6. The process of claim 2 wherein the catalyst comprises a complex of the formula MLₘXₙ wherein M is a platinum group metal, L is a bidiazine ligand, optionally substituted with one or more hydrocarbyl groups or substituted hydrocarbyl groups, or a substituent selected from —SO₃H, fluoride, or chloride, or any mixture thereof, X is an oxidation-resistant anion selected from halide, hydroxide, sulfate, bisulfate, triflate, nitrate and phosphate or the conjugate anion base of the strong acid employed, m is 1 or 2 and n is an integer of from 1 to 8.

7. The process of claim 6 wherein M is platinum and L is a bidiazine ligand of the formula: 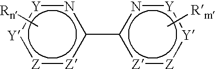
wherein Y, Y′, Z and Z′ are nitrogen or carbon with the proviso that one of Y, Y′, Z and Z′ must be nitrogen and the remainder of Y, Y′, Z and Z′ must be carbon, R and R′ are hydrogen, hydrocarbyl, substituted hydrocarbyl, fluoride or chloride or —SO₃H and m′ and n′ each are 0, 1, 2 or 3.

8. The process of claim 6 or 7 wherein the catalyst additionally comprises a co-catalyst selected from an inorganic salt of tellurium and antimony or mixtures thereof in intimate admixture with the catalyst complex.

9. The process of claim 6 wherein the bidiazine ligand is a 2,2′-bipyrimidine and X is a halide selected from chlorine, bromine and iodine.

10. The process of claim 9 wherein the platinum group metal is platinum.

11. The process of claim 10 wherein the catalyst additionally comprises a co-catalyst comprising a tellurium halide salt in intimate admixture with the catalyst complex.

12. The process of claims 1, 2, 6, 7 or 11 wherein the oxidizing agent is selected from the group consisting of HNO₃, perchloric acid, hypochlorites, peroxy compounds (H₂O₂, CH₃CO₃H, K₂S₂O₈), O₂ or O₃, SO₃, NO₂, H₂SO₄, cyanogen, quinones, halogens, selenium cations, tellurium cations and other oxidizing substances with redox potentials greater than 0.3 volts.

13. The process of claim 12 wherein the acid is selected from the group consisting of HNO₃, H₂SO₄, CF₃CO₂H, CF₃SO₃H, H₃PO₄, HCl, HF, HPAs (heteropolyacids), B(OH)₃, (CF₃SO₂)₂HN, (CF₃SO₂)₃CH or the like, anhydrides of these acids such as H₄P₂O₇, H₂S₂O₇ or the like and mixtures of two or more of these acids and anhydrides and mixtures of acids with Lewis acids such as CH₃CO₂H/BF₃, H₃PO₄/BF₃, H₃PO₄/SbF₅, HF/BF₃.

14. The process of claims 1, 2, 6, 7 or 11 wherein the lower alkane is selected from methane, ethane or propane.

15. The process of claim 14 wherein the lower alkane is methane.

16. The process of claim 11 wherein the oxidizing agent is selected from SO₃, H₂SO₄ and O₂.

17. The process of claim 16 wherein the acid is H₂SO₄.

18. The process of claim 17 wherein the oxidizing agent is H₂SO₄.

19. The process of claim 18 wherein the lower alkane is methane or ethane.

20. The process of claim 19 wherein the lower alkane is methane.

21. The process of claim 7 or 9 wherein the catalyst is prepared in situ by mixing a platinum compound, a bidiazine compound and an inorganic salt containing the oxidation-resistant anion in the strong acid prior to contacting the lower alkane reactant.

22. The process of claim 21 wherein the strong acid is H₂SO₄.

23. The process of claim 22 wherein the inorganic salt is a metal halide containing an anion selected from chloride, bromide or iodide.

24. The process of claim 23 wherein a co-catalyst comprising a tellurium halide salt is also added to the strong acid before contact with the lower alkane reactant.

25. The process of claim 24 wherein the lower alkane is selected from methane, ethane or propane.

26. The process of claim 25 wherein the lower alkane reactant is methane.

27. A catalyst composition comprising a catalytically active platinum group metal/ligand complex of the formula MLₘXₙ wherein M is a platinum group metal, L is a bidiazine ligand, optionally substituted with one or more hydrocarbyl groups or substituted hydrocarbyl groups, or a substituent selected from —SO₃H and fluoride or chloride or any mixture thereof, X is an oxidation resistant anion selected from halide, hydroxide, sulfate, bisulfate, nitrate and phosphate, m is 1 or 2 and n an integer of from 1 to 8.

28. The catalyst composition of claim 27 wherein the platinum group metal is selected from platinum or palladium.

29. The catalyst composition of claim 28 wherein the platinum group metal is platinum.

30. The catalyst composition of claim 29 wherein X is a halide selected from chloride, bromide or iodide.

31. The catalyst composition of claim 29 wherein L is a bidiazine ligand of the formula: 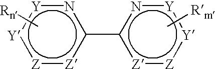
wherein Y, Y′, Z and Z′ are nitrogen or carbon with the proviso that one of Y, Y′, Z and Z′ must be nitrogen and the remainder of Y, Y′, Z and Z′ must be carbon, R and R′ are hydrogen, hydrocarbyl, substituted hydrocarbyl, fluoride or —SO₃H and m′ and n′ each are 0, 1, 2 or 3.

32. The catalyst composition of claim 27 or 31 wherein the catalyst additionally comprises a co-catalyst selected from an inorganic salt of tellurium and antimony or mixtures thereof in intimate admixture with the catalyst complex.

33. The catalyst composition of claim 31 wherein the bidiazine ligand is a 2,2′-bipyrimidine.

34. The catalyst composition of claim 33 wherein the catalyst additionally comprises a co-catalyst which is an inorganic salt of tellurium in intimate admixture with the catalyst complex.

35. The catalyst composition of claim 34 wherein the inorganic salt of tellurium is a tellurium halide selected from tellurium chloride, tellurium bromide or tellurium iodide.

36. The catalyst composition of claim 27 dissolved in a strong acid solvent.

37. The catalyst composition of claim 36 wherein the strong acid is H₂SO₄.

38. The catalyst composition of claim 36 wherein the catalyst complex is prepared by mixing a platinum group metal compound, a bidiazine ligand and an inorganic salt containing the oxidation resistant anion in the strong acid solvent.

39. The catalyst composition of claim 27 wherein the catalyst additionally comprises a co-catalyst which is an inorganic salt of tellurium or antimony or mixture thereof in intimate admixture with the catalyst complex.

40. The process of claim 1, 2, 6, 7, 9 or 11 wherein the lower alkyl ester obtained by partial oxidation of the lower alkane is subsequently reacted with a nucleophile to afford a functionalized derivative of the lower alkane.

41. The process of claim 40 wherein the nucleophile comprises a compound of the formula H-Y wherein Y is (OH, SH, Cl, Br, I, NH₂, or CN).

42. The process of claim 41 wherein the nucleophile is H₂O and the functionalized derivative is a mono- or poly-hydric alcohol derivative of the lower alkane starting material.

43. The process of claim 41 wherein the nucleophile is H₂S and the functionalized derivative is an alkyl thiol derivative of the lower alkane starting material.

44. The process of claim 42 wherein the lower alkane is methane and the functionalized derivative is methanol.

45. The process of claim 1, 2, 6, 7, 9 or 11 wherein the lower alkyl ester obtained by partial oxidation of the lower alkane is converted to a higher molecular weight hydrocarbon by (a) reacting the lower alkyl ester with a nucleophile to afford a functionalized derivative of the lower alkane, and (b) catalytically converting the functionalized derivative of the lower alkane to a higher molecular weight hydrocarbon.

46. In a process for the conversion of a lower alkane feed stream into comparatively higher molecular weight hydrocarbons, wherein the lower alkane feed stream is catalytically oxidized with an oxidizing agent in acidic media to produce an ester and the ester so obtained is then reacted with a nucleophile to yield a functionalized intermediate followed by catalytic conversion of the functionalized intermediate to a higher molecular weight hydrocarbon, the improvement which comprises employing a catalyst in the catalytic oxidation comprising a catalytic amount of a platinum group metal stabilized with a heteroatom-containing ligand which forms a mono-dentate or poly-dentate ligand complex with the platinum group metal, said complex being stable in the acidic media for at least about ten minutes at temperatures of about 180° C.
